(19) United States
(12) Patent Application Publication
(10) Pub. No.: US 2005/0169862 A1
(43) Pub. Date: Aug. 4, 2005

(54) Composition and method for the treatment of keratin fibers

(76) Inventor: Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/033,119

(22) Filed: Jan. 12, 2005

Related U.S. Application Data
(60) Provisional application No. 60/546,968, filed on Feb. 24, 2004.

(30) Foreign Application Priority Data
Jan. 29, 2004 (FR) 04 00854

(51) Int. Cl. A61K 7/09

(52) U.S. Cl. 424/70.2

(57) ABSTRACT

A method for treating keratin fibers by applying thereto a composition obtained by a process in which steam is percolated under a pressure of at least 3 bar through at least one oxidizing agent and/or at least one reducing agent, in solid or pasty form. The composition so made.

### REFERENCE TO PRIOR APPLICATIONS

This application claims priority to U.S. provisional application 60/546,968 filed Feb. 24, 2004, and to French patent application 0400854 filed Jan. 29, 2004, both incorporated herein by reference.

### FIELD OF THE INVENTION

In a preferred embodiment the present invention relates to a composition that may be obtained by the percolation of steam through a reducing and/or oxidizing agent, and its use in the treatment of keratin fibers, particularly human keratin fibers such as hair. A method of treatment for keratin fibers using the invention composition is also described.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In cosmetics, keratin fibers are normally bleached using compositions referred to as oxidizing compositions because they contain one or more agents capable of oxidizing the melanin in the hair and hence of solubilizing it for the total or partial removal thereof, and compositions referred to conversely as reducing compositions because they contain one or more reducing agents, such as ascorbic acid, sulphites and sulphinates, which are more especially intended for the bleaching of hair which has previously been dyed with exogenous pigments (stripping).

Furthermore, for hair perming, it is customary to apply to the hair, which has first been stretched, e.g. with curlers if curls are the desired waving, a composition containing one or more reducing agents so as to induce the opening of the disulphide bridges formed by the cysteine residues in the hair keratin, and then, generally after rinsing, reoxidize the hair to set the waving.

However, cosmetic treatment compositions containing such reducing and oxidizing agents are generally aqueous compositions in which said agents have to be solubilized. The efficacy of these compositions is reduced by the lack of solubility of these agents. In addition, this solubility criterion reduces the number of water-insoluble reducing and oxidizing agents which can be used for the cosmetic treatment of keratin fibers. This is particularly the case of high-melting compounds.

Furthermore, these reducing agents are intrinsically sensitive to oxidation and their stabilization in aqueous solution can pose problems. These instability problems are even more exaggerated if these reducing agents are esters sensitive to hydrolysis.

### DETAILED DESCRIPTION OF THE PREFERRED EMBODIMENTS

There has now been discovered, surprisingly, a process for the preparation of a composition for the treatment of keratin materials which affords, in a very short time (for example less than 2 minutes), compositions whose concentrations of reducing and oxidizing agents can vary as required, and which in preferred embodiments contain no preservatives, the resultant compositions overcoming the solubility problems referred to above.

The invention process is simple to carry out. For example, a fluid, at a temperature preferably greater than or equal to 30° C., particularly preferably ranging from 30° to 150° C. and very particularly preferably ranging from 40° C. to 120° C., is passed under pressure, for a short period of time (e.g., less than 1 minute, including 45, 30, and 15 sec), through at least one reducing agent and/or at least one oxidizing agent in solid or pasty form, preferably in solid form and particularly preferably in pulverulent form.

This process also makes it possible to use in anhydrous form reducing and/or oxidizing agents that are unstable in aqueous compositions, either because they react with water or because they react in aqueous solution with compounds which do not react with them in an anhydrous composition.

The compositions prepared by this process may have a limited storage stability, which is not a disadvantage here since the process provides a ready-to-use composition that is intended to be used quickly after it has been prepared, for example within 5 minutes of its preparation, especially after cooling to a cosmetically acceptable temperature preferably below 60° C. The composition can also be used up to one week after its preparation, or more, depending on the degradation rate of the reducing and/or oxidizing agent used.

In view of the very short preparation time, the invention compositions can be prepared “on demand” by mixing different cosmetically active compounds according to the desired cosmetic properties.

In another embodiment, as the reducing and/or oxidizing agents can be packaged in a ready-to-use device, the concentrations of the agents in solution do not need to be determined beforehand, thereby limiting the measurement errors made by the user.

The process makes it possible to avoid the use of multi-compartment bottles, making the process particularly economic and safer for the user.

Moreover, the process allows a reducing agent and an oxidizing agent to be combined, which is not possible in aqueous solution.

The resulting composition can be used on its own or in a mixture with another composition.

An additional advantage of this preparative process is that it provides compositions which impart good cosmetic properties. In particular, the keratin fibers treated with a composition based on reducing agents that has been obtained by the process according to the invention have improved cosmetic properties, especially a lasting smoothness, texture and body. For example, compositions based on oxidizing agents that are obtained by the process according to the invention strip and bleach the keratin fibers in a satisfactory and lasting manner.

The invention therefore relates a composition obtained by a process comprising a step in which a fluid is percolated under a pressure of at least 3 bar through at least one oxidizing agent and/or at least one reducing agent, in solid or pasty form, for the treatment (e.g., bleaching, perming, etc.) of keratin fibers.

It further relates to the use of the invention compositions for the treatment (e.g., bleaching, perming, etc.) of keratin fibers by application of the invention composition to fibers to be treated.

The invention further relates to a packaging device for carrying out the preparative process of the present invention.

Further subjects, characteristics, features and advantages of the invention will become even more clearly apparent from the description of highly preferred embodiments and Example which follow.

Hereafter, unless indicated otherwise, the limits defining a range of values are included within said range.

According to the invention, the process for the preparation of a composition for the cosmetic treatment of keratin fibers comprises a step in which fluid, preferably at a temperature advantageously greater than or equal to 30° C., preferably ranging from 30 to 150° C. and particularly preferably ranging from 40 to 120° C., is percolated under a pressure of at least 3 bar (3.10⁵ Pa) through at least one reducing agent and/or at least one oxidizing agent, in solid or pasty form.

Percolation is a movement of fluid through a porous medium that allows the fluid to pass through under the action or effect of pressure.

The fluid can consist of steam optionally accompanied by liquid water, or of one or more cosmetically acceptable liquid and/or gaseous solvents, especially organic solvents, or of a mixture of steam, optionally accompanied by liquid water, and one or more cosmetically acceptable liquid and/or gaseous solvents. Preferably, the fluid comprises at least steam possibly accompanied by liquid water; particularly preferably, it is steam possibly accompanied by liquid water.

Examples of organic solvents which may be mentioned are C₁-C₄ lower alcohols such as ethanol and isopropanol, polyols and polyol ethers such as 2-butoxyethanol, propylene glycol, propylene glycol monomethyl ether and diethylene glycol monoethyl ether and monomethyl ether, aromatic alcohols such as benzyl alcohol or phenoxyethanol, and mixtures thereof.

“Water-insoluble compound” is understood as meaning any compound which, at a concentration greater than or equal to 0.1% by weight in water at 25° C., does not form a transparent isotropic solution to the naked eye.

The reducing agent and/or oxidizing agent are in solid or pasty form, preferably in solid form and particularly preferably in pulverulent form.

It is possible to use the reducing agent by itself, the oxidizing agent by itself, or both of them together. In all instances more than one agent may be present—i.e., mixtures of reducing agents may be used, mixtures of two reducing agents and one oxidizing agent may be used, etc.

In terms of the present invention, “pasty form” is understood as meaning a consistency intermediate between a solid phase and a liquid phase. The viscosity of this pasty phase is preferably greater than 0.1 Pa·s and particularly preferably greater than 1 Pa·s at 25° C. with a shear rate of 10 s⁻¹.

The keratin fibers are more particularly human keratin fibers, for example eyelashes, eyebrows and hair.

The percolation process used in the present invention can be carried out by means of a conventional device for generating a fluid under pressure at a temperature preferably greater than or equal to 30° C., particularly preferably of between 30 and 150° C. and very particularly preferably of between 40 and 120° C. Such a device comprises a pressure-resistant chamber equipped with a heating unit, and a circuit for transporting the fluid to the reducing and/or oxidizing agent used in the invention.

In another embodiment, the device further comprises a liquid reservoir and a pump for transporting the liquid(s) to the chamber.

The liquid contained in the reservoir is either water, or a cosmetically acceptable solvent, or a mixture of several cosmetically acceptable solvents, or a mixture of water and one or more cosmetically acceptable solvents. Preferably, the liquid comprises at least water; particularly preferably, it is water.

A particularly useful device for carrying out the process of the present invention is a coffee machine of the “espresso” type. Such machines are well known in the art. These machines are described e.g. in patents AT 168405, U.S. Pat. No. 2,688,911, DE 32433870 and IT 1265636.

In one particular embodiment of the invention, the percolation step is carried out with a fluid at a temperature preferably greater than or equal to 30° C., particularly preferably of between 30 and 150° C. and very particularly preferably of between 40 and 120° C., under a pressure of between 3 and 30 bar, or of at least 4 bar, preferably greater than 10 bar and particularly preferably of between 10 and 30 bar.

The reducing and oxidizing agent(s) as described below, preferably in solid or pasty form, can be used directly in the device for generating the fluid under pressure, in a receptacle intended for this purpose. They can also be packaged in a particular device for packaging a cosmetic composition, comprising a closed housing delimited by at least one wall that is at least partially permeable to the fluid under a pressure of at least 3 bar, the composition containing at least one reducing agent and/or oxidizing agent. Such devices are described e.g. in patent applications WO 00/56629, EP 512470, WO 99/003753 and U.S. Pat. No. 5,897,899.

In one particular embodiment, the housing is delimited by two sealed sheets. In another embodiment, the housing is delimited by a small boat-shaped receptacle closed with a lid.

These devices can be made of woven or non-woven materials, a plastic, a plant material, for example cellulose, a metal such as aluminium, or a composite. Such devices are described e.g. in patent applications WO 00/56629, EP 512470 and WO 99/003753.

The reducing agent(s) which can be used in the process of the present invention are selected especially from those generally used in the field of the bleaching and perming of keratin fibers, such as alkali metal, alkaline earth metal or ammonium sulphites and/or bisulphites or, preferably, thiols. Among these reducing agents, those most commonly used are cysteine and its various derivatives (especially N-acetylcysteine), cysteamine and its various derivatives (especially its C₁-C₄ acyl derivatives such as N-acetylcysteamine or N-propionylcysteamine), thiolactic acid and its esters (especially glycerol monothiolactate), thioglycolic acid and its esters, especially glycerol or glycol monothioglycolate, diammonium dithiodiglycolate or ammonium thioglycolate, and thioglycerol. The following reducing compounds may also be mentioned: N-mercaptoalkylamides of sugars, such as N-(2-mercaptoethyl)gluconamide, β-mercapto-propionic acid and its derivatives, thiomalic acid, pantetheine, the N-(mercaptoalkyl)-ω-hydroxyalkylamides described in patent application EP-A-354 835 and the 4-(N-monoalkylmercapto or N,N-dialkylmercapto)butyramides described in patent application EP-A-368 763, the aminomercaptoalkylamides described in patent application EP-A-432 000 and the alkylaminomercapto-alkylamides described in patent application EP-A-514 282, and the mixture of 2-hydroxypropyl thioglycolate (2/3) and 2-hydroxy-1-methylethyl thioglycolate (67/33) described in patent application FR-A-2 679 448.

The reducing agents can also be selected from reductones such as ascorbic acid, erythorbic acid, their salts and their esters, and also from sulphinates such as those described in patent applications FR 2813016 and FR 2814948, particularly hydroxymethane-sulphinic acid and its salts. These reductones and sulphinates can be used particularly in stripping processes.

The reducing agent can also be an ester sensitive to hydrolysis.

The preferred reducing agents are selected especially from thioglycolic acid and its esters, cysteamine and cysteine. The particularly preferred reducing agent in the invention is thioglycolic acid or one of its esters, especially such as glycerol or glycol monothioglycolate or ammonium thioglycolate, or cysteine.

The oxidizing agent(s) can be selected from urea peroxides, bromates such as alkali metal bromates, per salts, mixtures of alkali metal bromates and a per salt, iodates, periodates, chlorates, perchlorates, chlorites, perchlorites, permanganates, especially those of alkali metals, and strontium peroxide.

The reducing and oxidizing agent(s) of the invention can be used in a mixture with one or more solid or pasty adjuvants, preferably pulverulent adjuvants. The adjuvants can be selected from clays, salts, anionic, non-ionic, cationic or zwitterionic surfactants, natural or synthetic thickeners, optionally modified starch, glass beads, silica, nylon, alumina, titanium dioxide, zeolites, polymethyl methacrylate (PMMA), chitosan, maltodextrin, cyclodextrin, monosaccharides or disaccharides such as glucose, sucrose, sorbitol or fructose, zinc oxide, zirconium oxide, particles of a resin such as silicone or silicabades, talcum, polyaspartic acid, borosilicates, especially calcium borosilicate, polyethylene, cotton, polytetrafluoroethylene (PTFE), cellulose and its derivatives, superabsorbent compounds, magnesium carbonate or calcium carbonate, maize seeds, polydimethylsiloxane gums, polyacrylamide, porous hydroxyapatite, silk, collagen, sawdust, bladderwrack powder, flours or extracts of wheat, rice, pea, lupin, soya or barley, crosslinked polyvinyl-pyrrolidone, calcium alginate, active charcoal, poly(vinylidene chloride/acrylonitrile) particles, especially those marketed under the general name “Expancel®” by AKZO NOBEL under the specific references “Expancel® WE” or “DE” Expancels, and mixtures thereof.

If one or more adjuvants are present, the reducing and/or oxidizing agent(s) of the invention is (are) present in an amount ranging preferably from 0.5 to 99% by weight, particularly preferably from 1 to 80% by weight and very particularly preferably from 2 to 60% by weight, based on the total weight of reducing and/or oxidizing agent(s) and adjuvant(s).

The plants or plant extracts used may be subjected, prior to percolation, to a treatment such as roasting, cryogrinding or lyophilization.

Apart from the reducing and/or oxidizing agent(s) and the component(s) of the fluid, namely the water and/or the cosmetically acceptable solvent(s), the composition for the cosmetic treatment of keratin materials which is obtained by the process of the invention optionally contains all or part of the adjuvant(s) present in the solid or pasty mixture.

The plants or plant extracts used, through which steam passes under pressure, may have undergone a prior treatment such as torrefaction, cryocomminution or lyophilization.

The invention further relates to a composition obtainable by the percolation process, the particularly preferred composition being devoid of preservatives.

The preparative process of the invention provides a composition for the cosmetic treatment of keratin fibers which can be applied directly to the keratin fibers or mixed with a cosmetically acceptable medium, or alternatively at least one additive conventionally used in cosmetics may be added thereto by an operator.

It is also possible to mix at least two compositions obtained by the process of the invention.

The composition for the cosmetic treatment of keratin fibers which may result from one or more mixing operations and/or addition operations indicated above will hereafter be called the final cosmetic treatment composition or final composition.

One particular embodiment of the invention consists in applying the resulting composition by means of a device that does not require human intervention and is optionally equipped with a cooling means.

The amount of reducing and/or oxidizing agent(s) present in the final composition is generally preferably between about 0.001 and 50% by weight, more preferably between 0.005 and 30% by weight and particularly preferably between 0.01 and 20% by weight, based on the total weight of the final composition.

If the cosmetic treatment composition obtained by the process of the present invention is mixed with a cosmetically acceptable medium, such a medium generally contains water or a mixture of water and at least one organic solvent for solubilizing the compounds which would not be sufficiently water-soluble.

Examples of organic solvents which may be mentioned are C₁-C₄ lower alcohols such as ethanol and isopropanol, polyols and polyol ethers such as 2-butoxyethanol, propylene glycol, propylene glycol monomethyl ether and diethylene glycol monoethyl ether and monomethyl ether, aromatic alcohols such as benzyl alcohol or phenoxyethanol, and mixtures thereof.

Preferably, the solvents are present in proportions preferably of between about 1 and 40% by weight and particularly preferably of between about 5 and 30% by weight, based on the total weight of the final cosmetic treatment composition.

Additives (adjuvants) can also be added to the invention compositions. Examples of such adjuvants include anionic, cationic, non-ionic, amphoteric or zwitterionic surfactants or mixtures thereof, anionic, cationic, non-ionic, amphoteric or zwitterionic polymers or mixtures thereof, mineral or organic thickeners, particularly associative thickeners of anionic, cationic, non-ionic and amphoteric polymers, antioxidants, penetrating agents, sequestering agents, perfumes, buffers, dispersants, conditioners, e.g. silicone oils, film-forming agents, preservatives and opacifiers, as well as oils, waxes, gums and coloured or nacreous pigments.

The above additives are generally present in an amount per additive of between 0.01 and 20% by weight, based on the weight of the final composition.

Of course, those skilled in the art will take care to preferably choose this or these optional complementary compounds in such a way that the advantageous properties intrinsically attached to the compositions according to the invention are unaffected or substantially unaffected by the envisaged addition(s).

The pH of the final composition is generally preferably between about 3 and 12 and more preferably between about 5 and 11. It can be adjusted to the desired value with, for example, acidifying or alkalizing agents normally used in cosmetics, or alternatively with conventional buffer systems.

Examples of acidifying agents which may be mentioned are mineral or organic acids such as hydrochloric acid, orthophosphoric acid, sulphuric acid, carboxylic acids, like acetic acid, tartaric acid, citric acid or lactic acid, and sulphonic acids.

Examples of alkalizing agents which may be mentioned are aqueous ammonia, alkali metal carbonates, alkanolamines, such as mono-, di- and triethanolamines, and derivatives thereof, sodium and potassium hydroxides and the compounds of formula (II) below: 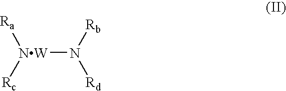
in which W is a propylene radical optionally substituted by a hydroxyl group or a C₁-C₄ alkyl radical, and Rₐ, R_{b}, R_{c} and R_{d}, which are identical or different, are a hydrogen atom, a C₁-C₄ alkyl radical or a C₁-C₄ hydroxyalkyl radical.

The compositions of the invention can be presented in diverse forms, including for example in the form of liquids, creams, mascara or gels or in any other form appropriate for treating keratin fibers and especially human hair.

The invention compositions can be used e.g. as a shampoo, an after-shampoo, a rinsed or unrinsed conditioner, a deep conditioning mask, a shower gel, a lotion or a cream for the treatment of keratin materials.

The present invention further relates to a method of cosmetic treatment for keratin materials which comprises preparing a cosmetic treatment composition by the process as described above and applying it to the keratin materials, for example by means of an operator or by means of a device that does not require human intervention. The application time can vary for example between 15 seconds and 1 hour.

Before application, the composition obtained by the process of the invention can be mixed with a cosmetically acceptable medium and/or with one or more additives, as described above.

Another embodiment of the invention is preparing at least two cosmetic treatment compositions by the process of the invention, mixing them, optionally adding a cosmetically acceptable medium and/or one or more additives conventionally used in cosmetics, as described above, and then applying the resulting final composition to the keratin materials.

It should be pointed out that the method of cosmetic treatment for keratin materials according to the invention can include carrying out two successive steps.

Thus, more particularly within the framework of a perming method, it is possible to apply a composition comprising at least one reducing agent, obtained by the process as defined above, to keratin fibers, the latter being stretched beforehand (curlers or smoothing). Then, after an appropriate contact time, a composition comprising at least one oxidizing agent, obtained by the process as defined above, can be applied, optionally after the fibers have been rinsed. After an appropriate contact time, the fibers may be rinsed, shampooed, rinsed again and finally dried or left to dry.

The Examples below are intended to illustrate the present invention, but not limit it.

### EXAMPLE 1

The following ingredients are mixed in the indicated proportions in % by weight, based on the total weight of the solid mixture:

| | |
|---|---|
| L-cysteine marketed by AJINOMOTO | 50% |
| maltodextrin sold under the trade name | 20% |
| GLUCIDEX 2 by ROQUETTE | |
| ammonium bicarbonate marketed by RHODIA | 30% |

5 g of this mixture are placed in an espresso machine. Steam is then passed through it until a composition (A) having a final volume of 50 ml is obtained.

Application can then be facilitated by adding one part by weight of an aqueous composition (B), containing 1% by weight of hydroxyethyl cellulose, to two parts by weight of composition (A).

This gives a perming composition ready for application to the hair.

The treated hair is lastingly waved and has a lasting body and texture.

### EXAMPLE 2

The following ingredients are mixed in the indicated proportions in % by weight, based on the total weight of the solid mixture:

| | |
|---|---|
| potassium persulphate | 20% |
| ammonium chloride | 30% |
| ammonium bicarbonate marketed by RHODIA | 10% |
| guar gum marketed under the name GUARGEL D/15 | 40% |
| by RHODIA | |

5 g of this mixture are placed in an espresso machine. Steam is then passed through it until a composition (A) having a final volume of 50 ml is obtained.

Application can then be facilitated by adding one part by weight of an aqueous composition (B), containing 1% by weight of hydroxyethyl cellulose, to two parts by weight of composition (A).

This gives a bleaching composition ready for application to the hair.

### EXAMPLE 3

The following ingredients are mixed in the indicated proportions in % by weight, based on the total weight of the solid mixture:

| | |
|---|---|
| carbamide peroxide marketed by PEROXID CHEMIE | 10% |
| erythorbic acid marketed by PFIZER | 10% |
| ammonium bicarbonate marketed by RHODIA | 60% |
| maltodextrin marketed under the name | 20% |
| GLUCIDEX 2 by ROQUETTE | |

5 g of this mixture are placed in an espresso machine. Steam is then passed through it until a composition (A) having a final volume of 50 ml is obtained.

Application can then be facilitated by adding one part by weight of an aqueous composition (B), containing 1% by weight of hydroxyethyl cellulose, to two parts by weight of composition (A).

This gives a bleaching composition ready for application to the hair.

The above written description of the invention provides a manner and process of making and using it such that any person skilled in this art is enabled to make and use the same, this enablement being provided in particular for the subject matter of the appended claims, which make up a part of the original description and including the use of a composition obtained by a process comprising a step in which steam is percolated under a pressure of at least 3 bar through at least one oxidizing agent and/or at least one reducing agent, in solid or pasty form, for the treatment of keratin fibers. Preferred embodiments of the invention similarly fully described and enabled include a composition for the cosmetic treatment of keratin materials that is obtainable by the invention process.

As used herein, the phrases “selected from,” “selected from the group consisting of,” “chosen from,” and the like include mixtures of the specified materials.

Where compounds are described as, e.g., “ . . . cysteine and its derivatives . . . ” an alternate is “cysteine and cysteine compounds,” where both cysteine derivatives and cysteine compounds are understood to share a common core/structure with cysteine. Those of ordinary skill are able to identify “X” derivatives based on a knowledge of “X” and the structural similarity of the compounds.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for treating keratin fibers, comprising applying thereto a composition obtained by a process comprising a step in which steam is percolated under a pressure of at least 3 bar through at least one oxidizing agent and/or at least one reducing agent, in solid or pasty form.

2. The method of claim 1, wherein said method is a method for bleaching or perming hair.

3. The method according to claim 1, in which steam is percolated under a pressure of at least 3 bar through at least one reducing agent, wherein the reducing agent(s) is (are) selected from alkali metal, alkaline earth metal or ammonium sulphites and/or bisulphites, thiols, N-mercaptoalkylamides of sugars, β-mercaptopropionic acid and its derivatives, thiomalic acid and pantetheine.

4. The method according to claim 3, wherein the at least one reducing agent comprises at least one thiol selected from cysteine and its derivatives, cysteamine and its derivatives, thiolactic acid and its esters, thioglycolic acid and its esters, and thioglycerol.

5. The method according to claim 3, wherein the at least one reducing agent comprises at least one selected from thioglycolic acid and its esters, cysteamine and cysteine.

6. The method according to claim 5, wherein the reducing agent is selected from glycerol or glycol monothioglycolate, ammonium thioglycolate and cysteine.

7. The method according to claim 1, in which steam is percolated under a pressure of at least 3 bar through at least one reducing agent, wherein the reducing agent is an ester sensitive to hydrolysis.

8. The method according to claim 1, in which steam is percolated under a pressure of at least 3 bar through at least one oxidizing agent, wherein the oxidizing agent is selected from urea peroxides, bromates, per salts, mixtures of alkali metal bromates and a per salt, iodates, periodates, chlorates, perchlorates, chlorites, perchlorites, permanganates, and strontium peroxide.

9. The method according to claim 1, wherein the oxidizing agent and/or the reducing agent are present in a mixture with at least one adjuvant.

10. The method according to claim 9, wherein the adjuvant is selected from clays, salts, anionic, non-ionic, cationic or zwitterionic surfactants, natural or synthetic thickeners, optionally modified starch, glass beads, silica, nylon, alumina, titanium dioxide, zeolites, polymethyl methacrylate (PMMA), chitosan, maltodextrin, cyclodextrin, monosaccharides or disaccharides, zinc oxide, zirconium oxide, particles of a resin such as silicone or silicabades, talcum, polyaspartic acid, borosilicates, especially calcium borosilicate, polyethylene, cotton, polytetrafluoroethylene (PTFE), cellulose and its derivatives, superabsorbent compounds, magnesium carbonate or calcium carbonate, maize seeds, polydimethylsiloxane gums, polyacrylamide, porous hydroxyapatite, silk, collagen, sawdust, bladderwrack powder, flours or extracts of wheat, rice, pea, lupin, soya or barley, crosslinked polyvinylpyrrolidone, calcium alginate, active charcoal and poly(vinylidene chloride/acrylonitrile) particles.

11. The method according to claim 9, wherein the reducing agent and/or the oxidizing agent are present in an amount ranging from 0.5 to 99% by weight based on the total weight of reducing agent and/or oxidizing agent and adjuvant(s).

12. The method according to claim 1, wherein the percolation step is carried out with a fluid under a pressure of between 3 and 30 bars.

13. The method according to claim 1, wherein the percolation step is carried out with a fluid under a pressure of at least 10 bar.

14. The method according to claim 1, wherein the fluid steam is optionally accompanied by liquid water.

15. The method according to claim 1, wherein the fluid comprises one or more cosmetically acceptable liquid and/or gaseous organic solvents.

16. The method according to claim 1, wherein said composition is applied to the keratin fibers by a device that does not require human intervention.

17. A composition obtained by a process comprising a step in which steam is percolated under a pressure of at least 3 bar through at least one oxidizing agent and/or at least one reducing agent, in solid or pasty form

18. A device for packaging a cosmetic composition, comprising a closed housing delimited by at least one wall that is at least partially permeable to a fluid under a pressure of at least 3 bar, the housing comprising at least one reducing agent and/or at least one oxidizing agent in solid or pasty form.

19. The device according to claim 18, in which the housing is delimited by two sealed sheets.

20. The device according to claim 18, wherein the housing is delimited by a boat-shaped receptacle closed with a lid.
